(19) United States
(12) Patent Application Publication
(10) Pub. No.: US 2006/0142282 A1
(43) Pub. Date: Jun. 29, 2006

(54) Combination effective for the treatment of impotence

(75) Inventor: Michael G. Wyllie, Herne (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/339,919

(22) Filed: Jan. 25, 2006

Related U.S. Application Data
(62) Division of application No. 10/255,538, filed on Sep. 25, 2002.
Application No. 10/255,538 is a continuation of application No. 09/367,169, filed on Nov. 12, 1999, now abandoned.
(60) Provisional application No. 60/069,741, filed on Dec. 16, 1997.

(51) Int. Cl. A61K 31/5377; A61K 31/519; A61K 31/498

(52) U.S. Cl. 514/234.5; 514/249; 514/252.16; 514/252.17

(57) ABSTRACT

This invention relates to the treatment of erectile dysfunction with a combination of (1) a compound selected from α-adrenergic receptor antagonists, and (2) a compound selected from agents which elevate cGMP levels. Sildenafil or a pharmaceutically acceptable salt thereof is preferred as the cGMP PDE elevator. Also included are compositions and kits comprising such impotence treating compounds.

### FIELD OF THE INVENTION

This invention relates to the treatment of impotence comprising co-administering (1) an α-adrenergic receptor antagonist and (2) an agent which elevates cyclic guanosine 3′,5′-monophosphate (cGMP) levels. The combination is particularly suitable for the treatment of patients suffering from impotence or erectile dysfunction.

### BACKGROUND OF THE INVENTION

Impotence is the inability to obtain and/or sustain an erection sufficient for penetration of the vagina and/or intercourse. Thus, impotence is also referred to as “erectile insufficiency” or “erectile dysfunction”. It has been estimated that 10-12 million American men between the ages of 18 and 75 suffer from chronic impotence, with the great majority being over age 55.

The penis normally becomes erect when certain tissues, in particular the corpora cavernosa in the central portion of the penis, become engorged with blood, thereby causing them to become less flaccid, and in turn causing an erection. Impotence can result from psychologic disturbances (psychogenic), from physiologic abnormalities (organic) or from a combination of both. Thus, in some males erectile dysfunction may be due to anxiety or depression, with no apparent somatic or organic impairment. In other cases, erectile dysfunction is associated with atherosclerosis of the arteries supplying blood to the penis. In still other cases, the dysfunction may be due to venous leakage or abnormal drainage in which there is leakage from veins in the penis such that sufficient pressure for an erection can be neither obtained nor maintained. In still other cases, the dysfunction is associated with a neuropathy or due to nerve damage arising from, for example, surgery or a pelvic injury. Typically, multiple factors are responsible for impotence.

α-Adrenergic receptors (herein also referred to as “α-adrenoceptors” or as “α-receptors”) are specific protein recognition sites located in the peripheral and central nervous systems and other tissues throughout the body. Neurotransmitters such as norepinephrine control many physiologic functions via an action on these receptors and thereby transmit information between cells or influence biochemical processes within the cell. Many agents capable of modifying norepinephrine activity on α-adrenoceptors have been developed over the last 40 years.

Drugs active at α-adrenoceptors can be broken into two major classes, agonists and antagonists. Agonists, of which clonidine and naphazoline are examples, activate the receptor system in the same way as the endogenous neurotransmitters, norepinephrine and epinephrine. Antagonists, of which phenoxybenzamine and prazosin are examples, do not activate the receptor but block the actions of the endogenous neurotransmitters.

Different α-adrenoceptor types have been discovered over the years including α₁-adrenoceptors and α₂-adrenoceptors. These receptor types are now considered to be further subdivided into subtypes including 1A, 1B, 1D, 1H, 1L, 1N, 2A, 2B, and 2C.

α₂-Adrenoceptors located on nerve terminals, by an action dependent at least in part on neurotransmitter release, are known to reduce activity in the sympathetic nervous system and increase activity within the parasympathetic nervous system, particularly in the vagus nerve. In addition, α₂-adrenoceptors on other tissues in the body control platelet aggregation, lipolysis and metabolism. α₂-Adrenoceptor antagonists have been disclosed for a wide variety of therapies, including reversing the state of anesthesia (U.S. Pat. No. 5,636,204), for the treatment of glaucoma (U.S. Pat. No. 4,590,202), for the treatment of cognitive disorders such as endogenous depression, age dependent memory impairment, and Alzheimer's disease (U.S. Pat. No. 5,498,623), and for the treatment of numerous other neurodegenerative disorders (U.S. Pat. No. 5,281,607).

α₁-Adrenoceptors are known to mediate the contraction of arterial and venous smooth muscle. α₁-Adrenoceptor antagonists have been used widely as first line therapy for the treatment of hypertension and, more recently, for the symptomatic relief of benign prostatic hyperplasia, BPH. See Kenny et al., Exp. Opin. Invest. Drugs (1995) 4(10), pp 915-923. Some compounds which have α₁-adrenoceptor antagonist activity, such as phentolamine and trazodone are used to treat impotence, although the mechanism (or mechanisms) of promoting erectile function is not completely understood. Such compounds are believed to work at least in part through blocking the action of norepinephrine which, without being blocked, otherwise causes contraction of the cavernosal smooth muscle allowing venous blood to leave the penis, and thereby produces de-tumescence and flaccidity of the organ. Many such compounds have been delivered locally by intra-cavernosal injection and are often associated with complications such as priapism (prolonged and painful erection), pain and infection at the site of injection and, in the long term, tissue fibrosis. Apart from the obvious discomfort, there is an associated loss of spontaneity.

α-Adrenoceptors can also mediate a reduction in cavernosal smooth muscle contraction indirectly by reducing sympathetic nervous activity by central actions, such effect being known for trazadone, and certain centrally active α₂-receptor agonists such as clonidine, or by a direct action on the smooth muscle cells as exemplified by papaverine.

Agents which elevate cGMP levels are also well known and can work through any of several mechanisms. Agents which selectively inhibit an enzyme predominantly involved in cGMP breakdown, for example a cGMP phosphodiesterase (cGMP PDE), constitute one example. Other phosphodiesterases can also hydrolyze cGMP, and inhibitors of these enzymes including compounds such as rolipram, zaprinast and xanthine derivatives such as caffeine, theophylline and theobromine, can accordingly influence cGMP levels. Other compounds which increase cGMP levels can do so through different mechanisms including the activation of soluble guanylate cyclase or membrane-bound guanylate cyclase, either directly as in the case of atrial natriuretic peptide, or indirectly. Other compounds act to increase cellular cGMP levels by modulation of cytokines. Other classes of cGMP elevators include muscarinic agonists, which can elevate cGMP levels without altering phosphodiesterase activity. Some prostaglandins such as PGE₁ are also known cGMP elevators. Kanba et. al., J. Neurochem., Vol. 57, No. 6, 1991.

Cyclic guanosine 3′,5′-monophosphate phosphodiesterase (cGMP PDE) inhibitors are widely known as cardiovascular agents for the treatment of conditions such as angina, hypertension, and congestive heart failure. More recently cGMP PDE inhibitors have been found to be effective for the treatment of impotence, importantly by oral administration. See, for example, PCT/EP94/01580, published as WO 94/28902. It is believed that such compounds may manifest their therapeutic effects by achieving high cGMP levels through inhibiting phosphodiesterase, thereby relaxing and expanding cavernosal cells and blocking the outflow of blood from the penis.

### SUMMARY OF THE INVENTION

This invention provides a method of treating impotence (also known in the art and referred to herein as “male erectile dysfunction”), especially in humans, comprising co-administering to a patient in need of such treatment an effective mount of:

(1) a compound selected from α-adrenoceptor antagonists (herein also referred to as α-antagonists), and

(2) a compound which elevates cGMP levels (herein also referred to as a cGMP elevator).

Reference to a compound or agent within the scope of (1) or (2), above, such as to an α-antagonist and/or to a cGMP elevator, both in this disclosure and the appendant claims, shall at all times be understood to include all active forms of such agents, including the free form thereof (e.g., the free acid or base form) and also all pharmaceutically acceptable salts, prodrugs, polymorphs, hydrates, solvates, stereoisomers (e.g. diastereomers and enantiomers), and so forth. Active metabolites of either the α-antagonist or the cGMP elevator, in any form, are also included.

The α-antagonist can be selective for either α₁- or α₂-adrenoceptors, or it can be non selective, exhibiting antagonist activity at both α₁- and at α₂. Non selective antagonists are preferred. Antagonists selective for the α₁-adrenoceptor are more preferred. In the context of the known α₁-adrenoceptor subtypes, antagonists at 1A, 1B, 1D, 1H, 1N and 1L are equally preferred.

As the cGMP elevator, cGMP PDE inhibitors are preferred. cGMP PDE inhibitors which are selective for cGMP PDEs rather than cyclic adenosine 3′,5′-monophosphate phosphodiesterases (cAMP PDEs) and/or which are selective inhibitors of the cGMP PDEᵥ isoenzyme are particularly preferred. Such particularly preferred cGMP PDE inhibitors are disclosed in U.S. Pat. Nos. 5,250,534, 5,346,901, 5,272,147, and in the international patent application published as WO 94/28902 designating, inter alia, the U.S., each of which is incorporated herein by reference.

Preferred combinations of an α-adrenoceptor antagonist and a cGMP PDE elevator useful herein are “synergistic”, meaning that the therapeutic effect of co-administering compounds selected from (1) and (2) as defined above is greater than additive. Thus, co-administering both therapeutic agents produces an effect which is greater than the sum of the effects of each agent administered alone. Such synergy is advantageous in that it allows for each therapeutic agent typically to be administered in an amount less than if the combined therapeutic effects were additive. Thus, therapy can be effected for patients who, for example, do not respond adequately to the use of one component at what would be considered a maximal strength dose. Additionally, by administering the components in lower amounts relative to the case where the combined effects are additive, side effects such as priapism or pain at the site of injection can be minimized or avoided in many cases. Such synergy can be demonstrated by the tests disclosed below.

The synergy of such preferred combinations is provided as a further feature of the invention, and accordingly the invention provides a method for achieving a synergistic therapeutically effective level of impotence treatment, comprising co-administering to a mammal in need of such treatment

(1) an amount of a first compound selected from α-adrenoceptor antagonists; and

(2) an amount of a second compound selected from compounds which elevate cGMP levels;

wherein the amount of the first compound alone and the amount of the second compound alone are each insufficient to achieve the synergistic therapeutically effective level of impotence treatment, but wherein the combined effect of the amounts of the first and second compounds is greater than the sum of the levels of therapeutic effects of impotence treatment achievable with the individual amounts of the first and second compound.

Additional preferred combinations include those which can be taken “on demand”, as opposed to needing to be taken chronically. Such preferred combinations include those which modulate the sexual response such that the patient responds to sexual (e.g., visual) stimulation, as opposed to compositions which act by causing an erection in the absence of sexual stimulation.

Additional preferred combinations include those which are “fast acting”, meaning that the time taken from administration to the point at which the sexual response can be modulated is less than about two hours, preferably less than about one hour, more preferably on the order of a half hour or less, and even more preferably within 10 or 15 minutes.

“Co-administration” when used in this disclosure and the appendant claims, for example in referring to a combination of an α₁-antagonist and a cGMP PDE inhibitor, means that the individual components can be administered together as a composition if the route of administration for each component is the same. Thus the invention further provides a composition comprising

(1) a first compound, said first compound being selected from α-adrenoceptor antagonists;

(2) a second compound which elevates cGMP levels; and

(3) a pharmaceutically acceptable carrier.

A preferred group of compositions are synergistic. Such synergistic compositions, which are provided as a further feature of the invention, comprise

(1) an amount of a first compound selected from α-adrenoceptor antagonists;

(2) an amount of a second compound selected from compounds which elevate cGMP levels;

wherein the amount of the first compound alone and the amount of the second compound alone are each insufficient to achieve a synergistic therapeutically effective level of impotence treatment, but wherein the effect of a composition comprising said amounts of said first and second compounds is greater than the sum of the levels of therapeutic effects of impotence treatment achievable with the individual amounts of said first and second compound; and a pharmaceutically acceptable diluent or carrier.

“Co-administration” also includes administering each of compounds (1) and (2) separately but as part of the same therapeutic treatment program or regimen, and it is contemplated that separate administration of each compound, at different times and by different routes, will sometimes be recommended. Thus, the two compounds need not necessarily be administered at essentially the same time. In a preferred embodiment, administration is timed so that the peak pharmacokinetic effect of one compound coincides with the peak pharmacokinetic effect for the other. If co-administered separately, it is also preferred that both of compounds (1) and (2) be administered in an oral dosage form.

Reference herein to a “combination” is to the co-administration of a compound selected from (1) and a compound selected from (2), either as a composition or separately, e.g., by different routes of administration.

The invention further provides a method of treating impotence, especially in humans, comprising administering, to a male human in need of such treatment, an effective amount of doxazosin, or a pharmaceutically acceptable salt thereof. The doxazosin can be administered as the only active compound, i.e., it need not be co-administered with an α-antagonist, or with any other active compound, although it can be. It can be administered in an amount of from 0.01 to 50 mg per day, preferably from 0.5 to 10 mg per day, usually orally, or by other route of administration as described herein, as a composition comprising doxazosin and a pharmaceutically acceptable carrier as also described herein. Such compositions can also be employed for the treatment of female sexual dysfunction, as further disclosed below.

The compositions of this invention are also useful for the treatment of sexual dysfunction in female mammals, including humans. Thus the compositions are useful, for example, in the treatment of female sexual dysfunction including orgasmic dysfunction related to clitoral disturbances. As in the case of male mammals, compositions which are synergistic, which can be taken on demand, and which modulate the female sexual response are preferred. Preferred compounds, compositions, and combinations (e.g. of compounds for separate administration) for the treatment of female sexual dysfunction are the same as those disclosed herein for the treatment of male erectile dysfunction.

Methods for the treatment of female sexual dysfunction are analogous to those presented herein for the treatment of impotence or erectile dysfunction in male animals.

Since the present invention has an aspect that relates to the treatment of impotence or of female sexual dysfunction by treatment with a combination of compounds which may be co-administered separately, the invention also relates to combining separate pharmaceutical compositions in kit form. The kit comprises two separate pharmaceutical compositions: (1) a composition comprising a compound selected from α-adrenergic receptor antagonists, plus a pharmaceutically acceptable carrier or diluent; and (2) a composition comprising a compound selected from agents which elevate cGMP levels, plus a pharmaceutically acceptable carrier or diluent. The amounts of (1) and (2) are such that, when co-administered separately, the impotence condition or condition of female sexual dysfunction is treated and/or remediated. The kit comprises a container for containing the separate compositions such as a divided bottle or a divided foil packet, wherein each compartment contains a plurality of dosage forms (e.g., tablets) comprising (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising pharmaceutical composition (1), and the second (or more) tablet(s) comprising pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises

(1) a therapeutically effective amount of a composition comprising a compound selected from α-adrenergic receptor antagonists, plus a pharmaceutically acceptable carrier or diluent, in a first dosage form;

(2) a therapeutically effective amount of a composition comprising a compound selected from compounds which elevate cGMP levels, plus a pharmaceutically acceptable carrier or diluent, in a second dosage form; and

(3) a container for containing said first and second dosage forms.

An example of such a kit, alluded to above, is a so-called blister pack. Blister packs are well known in the packaging industry and are widely used for the packaging of pharmaceutical unit dosage forms such as tablets, capsules, and the like. Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. Tablet(s) or capsule(s) can then be removed via said opening.

It may be desirable to provide a memory aid on the kit, e.g., in the form of numbers next to the tablets or capsules whereby the numbers correspond with the days of the regimen during which the tablets or capsules so specified should be ingested. Another example of such a memory aid is a calendar printed on the card, e.g., as follows “First Week, Monday, Tuesday, . . . etc. . . . Second Week, Monday, Tuesday, . . . ”, etc. Other variations of memory aids will be readily apparent. A “daily dose” can be a single tablet or capsule or several pills or capsules to be taken on a given day. Also a daily dose of the first compound can consist of one tablet or capsule while a daily dose of the second compound can consist of several tablets or capsules and vice versa. The memory aid should reflect this.

Other pharmaceutical components may also be optionally included as part of the combinations useful in this invention so long as they do not interfere or adversely affect the effects of the α-antagonist/cGMP elevator combination.

A preferred combination is a cGMP PDE inhibitor and a selective α₂-antagonist.

A more preferred combination is a cGMP PDE inhibitor and a non-selective α-antagonist.

A still more preferred combination is a cGMP PDE inhibitor and a selective α₁-antagonist.

Preferred combinations further include (1) respectively, in ascending order of preference, an α₂-antagonist, a non-selective α-antagonist, or a selective α₁-antagonist; and (2) a cGMP PDE inhibitor that is selective for the PDEᵥ isoenzyme. Compounds selective for the PDEᵥ isoenzyme are disclosed and characterized, for example, in PCT/EP94/01580, published as WO 94/28902 and which designates, inter alia, the United States, and which is incorporated herein by reference.

Preferred cGMP PDE inhibitors include sildenafil which has the structure: 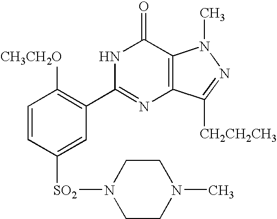
and pharmaceutically acceptable salts thereof, and the compound having the structure: 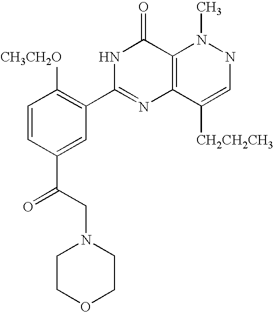
and pharmaceutically acceptable salts thereof. The second compound is disclosed, for example, in U.S. Pat. Nos. 5,272,147 and 5,426,107, both incorporated herein by reference.

A preferred pharmaceutically acceptable salt of sildenafil for use in this invention is the citrate salt, disclosed in co-pending U.S. application Ser. No. 08/944,546 filed Oct. 7, 1997 and incorporated herein by reference.

Also preferred are compounds disclosed in PCT/EP95/00183, published as WO 95/19978 designating, inter alia, the United States, and herein incorporated by refrence, said compounds having the formula 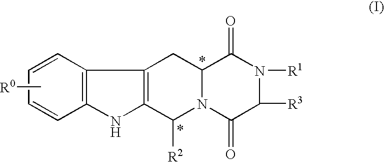
and salts and solvates thereof, in which:
R⁰ represents hydrogen, halogen or C₁₋₆alkyl,;
R¹ represents hydrogen, C₁₋₆alkyl, C₂₋₆alkenyl, C₂₋₆alkynyl, haloC₁₋₆alkyl, C₃₋₈cycloalkyl, C₃₋₈cycloalkylC₁₋₃alkyl, arylC₁₋₃alkyl or heteroarylC₁₋₃alkyl;
R² represents an optionally substituted monocyclic aromatic ring selected from benzene, thiophene, furan and pyridine or an optionally substituted bicyclic ring 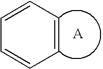
   attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; and
R³ represents hydrogen or C₁₋₃alkyl, or R¹ and R³ together represent a 3- or 4-membered alkyl or alkenyl chain.

A preferred subset of compounds having formula la (also disclosed in WO 95/19978) includes compounds of the formula 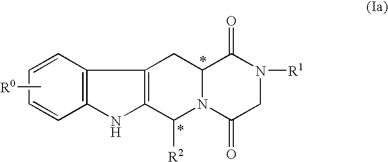
and salts and solvates thereof, in which:
R⁰ represents hydrogen, halogen or C₁₋₆alkyl;
   R¹ represents hydrogen, C₁₋₆alkyl, haloC₁₋₆alkyl, C₃₋₈cycloalkyl, C₃₋₈cycloalkyl-C₁₋₃alkyl, arylC₁₋₃alkyl or heteroarylC₁₋₃alkyl; and
R² represents an optionally substituted monocyclic aromatic ring selected from benzene thiophene, furan and pyridine or an optionally substituted bicyclic ring 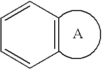
   attached to the rest of the molecule via one of the benene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen.

A specific compound within formulae (I) is:

(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione.

Preferred α-antagonists include doxazosin, terazosin, abanoquil, and prazosin, and the pharmaceutically acceptable salts thereof (especially doxazosin mesylate, terazosin hydrochloride, and prazosin hydrochloride), which are selective for α₁ adrenoceptors. Preferred specific combinations include any of these in combination with sildenafil or a pharmaceutically acceptable salt thereof, particularly the citrate salt. Most preferred are sildenafil citrate in combination with doxazosin mesylate or abanoquil mesylate.

Examples of additional α-antagonists include alfuzosin, indoramin, naftopidil, phentolamine, tamsulosin, trazodone, dapiprazole, phenoxybenzamine, idazoxan, efaroxan, and yohimbine, and also pharmaceutically acceptable salts thereof. Also useful are the rauwolfa alkaloids. Of these, phenoxybenzamine, phentolamine, trazodone, and dapiprazole are reported to be non-selective. Rauwolfa alkaloids, idazoxan, efaroxan and yohimbine are reported to be selective for α₂ receptors. The other specific compounds above are reported to be selective for α₁ receptors.

Further α-antagonists which are reported to be specific for α₁ include:

Recordati 15/2739 which has the structure

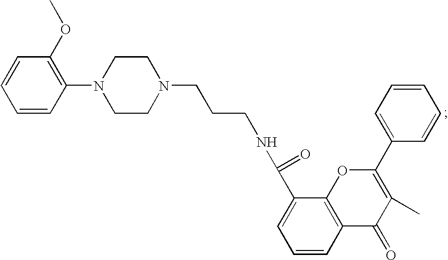

SNAP 1069 which has the structure

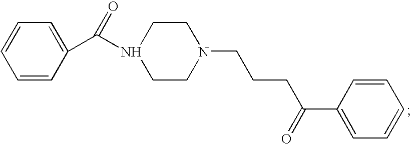

SNAP 5089 which has the structure

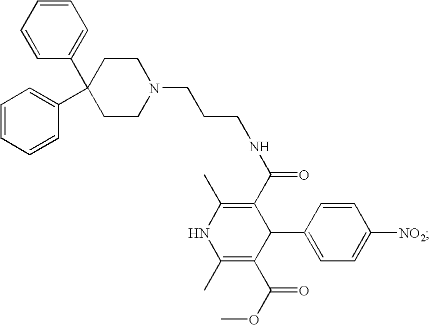

RS 17053 which has the structure

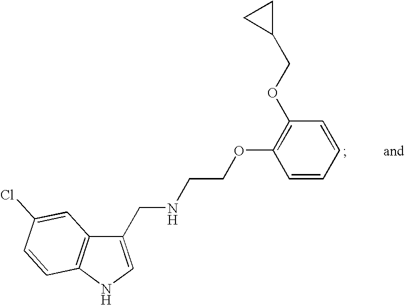

SL 89.0591 which has the structure

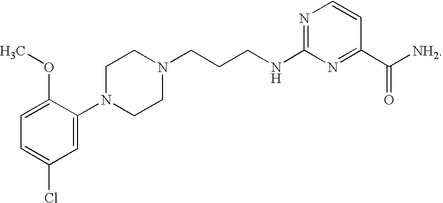

Specific combinations of an α-antagonist and a cGMP elevator useful in this invention include any adrenoceptor antagonist in combination with sildenafil. Combinations of sildenafil, especially sildenafil citrate, with an (α₁-selective antagonist, including any of those previously noted, are preferred.

### DETAILED DESCRIPTION

The cGMP PDE inhibitors useful in this invention as cGMP elevators may be widely chosen from among any of those already known to the art or subsequently discovered and/or hereafter developed. Suitable cGMP PDE inhibitors include those disclosed in any of the following US patents, all of which are incorporated herein by reference:

a 5-substituted pyrazolo[4,3-d]pyrimidine-7-one as disclosed in U.S. Pat. No 4,666,908;

a griseolic acid derivative as disclosed in any of U.S. Pat. Nos. 4,634,706, 4,783,532, 5,498,819, 5,532,369, 5,556,975, and 5,616,600;

a 2-phenylpurinone derivative as disclosed in U.S. Pat. No. 4,885,301;

a phenylpyridone derivative as disclosed in U.S. Pat. No. 5,254,571;

a fused pyrimidine derivative as disclosed in U.S. Pat. No. 5,047,404;

a condensed pyrimidine derivative as disclosed in U.S. Pat. No. 5,075,310;

a pyrimidopyrimidine derivative as disclosed in U.S. Pat. No. 5,162,316;

a purine compound as disclosed in U.S. Pat. No. 5,073,559;

a quinazoline derivative as disclosed in U.S. Pat. No. 5,147,875;

a phenylpyrimidone derivative as disclosed in U.S. Pat. No. 5,118,686;

an imidazoquinoxalinone derivative or its aza analog as disclosed in U.S. Pat. Nos. 5,055,465 and 5,166,344;

a phenylpyrimidone derivative as disclosed in U.S. Pat. No. 5,290,933;

a 4-aminoquinazoline derivative as disclosed in U.S. Pat Nos. 5,436,233 or 5,439,895;

a 4,5-dihydro-4-oxo-pyrrolo[1,2-a]quinoxaline derivative as disclosed in U.S. Pat. No. 5,405,847;

a polycyclic guanine derivative as disclosed in U.S. Pat. No. 5,393,755;

a nitogenous heterocyclic compound as disclosed in U.S. Pat. No. 5,576,322;

a quinazoline derivative as disclosed in U.S. Pat. No. 4,060,615; and

a 6-heterocyclyl pyrazolo[3,4-d]pyrimidin-4-one as disclosed in U.S. Pat. No. 5,294,612.

Other disclosures of cGMP PDE inhibitors include the following, all of which are herein incorporated by reference:

European patent Application (EPA) publication no. 0428268;

European patent 0442204;

International patent application publication no. WO 94/19351;

Japanese patent application 5-222000;

European Journal Of Pharmacology, 251, (1994), 1; and

International patent application publication no. WO 94/22855.

α-antagonists and salts thereof, in addition to those specifically identified above, have been widely disclosed in the patent literature, including U.S. Pat. Nos. 4,188,390, 4,026,894, 3,511,836, 4,315,007, 3,527,761, 3,997,666, 2,503,059, 4,703,063, 3,381,009, 4,252,721, and 2,599,000, each of which is incorporated herein by reference.

The α-antagonism of a compound, and therefore its suitability for use in the present invention, can be determined using a number of conventional assays in vitro. Suitable assays include those disclosed in U.S. Pat. No. 5,599,810 which employ rabbit aorta to determine α₁-adrenoceptor antagonist activity and guinea pig left atrium to determine α₂, and in U.S. Pat. No. 5,340,814 which employ rat brain cortex membranes to determine both α₁ and α₂ antagonist activity. Both of those patents are incorporated herein by reference

The cGMP PDE inhibition of a compound can also be determined by standard assays known to the art, for example as disclosed in U.S. Pat. No. 5,250,534, incorporated herein by reference. Compounds which are selective inhibitors of cGMP PDE relative to cAMP PDE are preferred, and determination of such compounds is also taught in U.S. Pat. No. 5,250,534. Particularly preferred are compounds which selectively inhibit the PDEᵥ isoenzyme, as disclosed in the aforementioned PCT/EP94/01580, published as WO 94/28902.

As disclosed above, individual compounds of the combinations useful in this invention will generally be administered separately, each by its own customary and known route, and in certain cases the routes of administration may be different. In a preferred embodiment, administration will generally be timed so that both the α-antagonist and the cGMP elevator both coincide, or nearly coincide, in reaching their maximum pharmacokinetic effect. The routes of administration can be any of those known to the art such as oral, parenteral via local injection intracavernosally or intraurethrally, or transdermal as by applying the active component in a gel or other such formulation topically to the penis. Each component can be formulated as known in the art, usually together with a pharmaceutically acceptable carrier or diluent, for example as a tablet, capsule, lozenge, troche, elixir, solution, or suspension for oral administration, in a suitable injectable vehicle for parenteral administration, or as a lotion, ointment or cream for topical application. In a preferred embodiment, the cGMP elevator and the α-antagonist are each co-administered orally, together or separately.

The exact dose of each component administered will, of course, differ depending on the specific components prescribed, on the subject being treated, on the severity of the impotence or of the female sexual dysfunction, on the manner of administration and on the judgment of the prescribing physician. Thus, because of patient-to-patient variability, the dosages given below are a guideline and the physician may adjust doses of the compounds to achieve the treatment that the physician considers appropriate for the patient, male or female. In considering the degree of treatment desired, the physician must balance a variety of factors such as the age of the patient and the presence of other diseases or conditions (e.g., cardiovascular disease). In general, the cGMP elevator will be administered in a range of from 0.5 to 200 mg per day, preferably 10 to 125 mg per day, more preferably 25-100 mg per day. The α-antagonist will generally be administered in an amount of from 0.01 mg to 50 mg per day, preferably from 0.5 to 10 mg per day. If the cGMP PDE elevator is a prostaglandin, it is generally administered intracavernosally by injection in an amount of from 1 ng to 100 μg or intraurethrally in an amount of 100 μg to 2 mg per day. Generally, the injected amount is in a volume which usually will not exceed 1 ml. The carrier or diluent is typically sterile physiological saline or another physiologically acceptable salt solution. Oral administration of prostaglandins is also feasible. Japanese Journal of Urology, 83(10):1655-1661, (1992).

As previously disclosed, the combination of cGMP PDE elevator and α-adrenoceptor antagonist can be administered as a composition. Thus, the compounds of this invention can be administered together in any conventional oral, parenteral, rectal or transdermal dosage form, usually also together with a pharmaceutically acceptable carrier or diluent.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

A combination of an α-antagonist and a cGMP elevator such as a cGMP PDE inhibitor can be tested in vivo in either a beagle dog or monkey model. The following description is with respect to monkeys, but those skilled in the art will easily recognize that the test applies equally and can be adapted to beagle dogs.

Mature adult male monkeys, typically either Cercopithecus aethiops (green monkey) or Macaca fasciculata (cynomologous) having a weight range of 4 to 8 kg are used. Animals are anesthetized with diazepam (2.5 mg), ketamine chloride (20 μg/kg i.m. supplemented as appropriate) and given the appropriate compound(s) dissolved in saline intracavernosally (0.3 ml). Animals are placed supine, the penis stretched out, and a rubber band placed around the root of the base as a tourniquet kept in place for three minutes after the injection. The solution is injected through a 27 G needle into one of the corpus cavernosa and 5, 10, 25, 30, 60, and 180 minutes later tumescence (increase in volume) and rigidity of the penis is estimated visually and by palpitation. To determine the threshold effect using the injectable solution a series of animals are used covering an appropriate dose range for the test compound or compounds. The threshold effect is determined for the test compound or compounds.

The combination of an α-antagonist and cGMP elevator can also be tested clinically, typically orally, in humans as well as in an animal model. Each component is administered singly at different times to a population of male patients, each component being administered in an amount which produces little or no response, typically less than a 50% response, as measured by the Rigiscan Clinical Evaluation parameters (see Kaneko et al., J. Urol. 136, 1026-1029 (1986); and Ogric et al., J. Urol., 154, 1356-1359 (1995) ) of rigidity and tumescence, in conjunction with the International Index of Erectile Function (IIEF) questionnaire which evaluates patient and partner satisfaction. By administering each component singly, it is meant that one component is administered, followed at a later time by the second component after having allowed an appropriate time for washout of the first component. After the washout period for each component administered singly, the components are co-administered in a manner such that both components co-operate pharmacokinetically, preferably such that the peak pharmacokinetic effect due to each coincides. Co-administration is evaluated according to the regiscan parameters mentioned above and by IIEF questionnaires, thereby providing a basis for comparison of the effects of co-administration with that for each single administration.

1.-30. (canceled)

31. A composition, comprising:
(1) a first compound selected from α-adrenoceptor antagonists;
(2) a second compound which elevates cGMP levels; and
(3) a pharmaceutically acceptable carrier.

32. A composition as defined in claim 31, wherein said cGMP elevator is a cGMP PDE inhibitor.

33. A composition as defined in claim 31, wherein said cGMP PDE elevator is a prostaglandin.

34. A composition as defined in claim 32, wherein said cGMP PDE inhibitor is selective for the cGMP PDEᵥ isoenzyme.

35. A composition as defined in claim 32, wherein said cGMP PDE inhibitor is sildenafil or a pharmaceutically acceptable salt thereof.

36. A composition as defined in claim 35, wherein said salt is the citrate salt.

37. A composition as defined in claim 32, wherein said cGMP PDE inhibitor has the structure 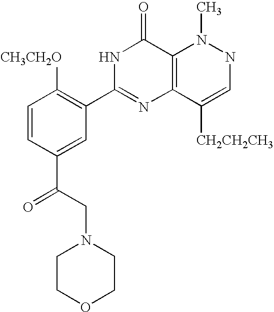
or is a pharmaceutically acceptable salt thereof.

38. A method as defined in claim 32, wherein said cGMP PDE inhibitor has the structure 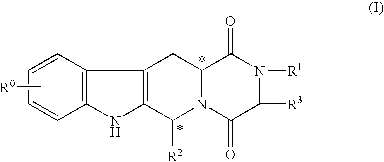
and salts and solvates thereof, in which:
R⁰ represents hydrogen, halogen or C₁₋₆alkyl,;
R¹ represents hydrogen, C₁₋₆alkyl, C₂₋₆alkenyl, C₂₋₆alkynyl, haloC₁₋₆alkyl, C₃₋₈cycloalkyl, C₃₋₈cycloalkylC₁₋₃alkyl, arylC₁₋₃alkyl or heteroarylC₁₋₃alkyl;
R² represents an optionally substituted monocyclic aromatic ring selected from benzene, thiophene, furan and pyridine or an optionally substituted bicyclic ring 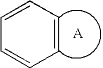
attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; and
R³ represents hydrogen or C₁₋₃alkyl, or R¹ and R³ together represent a 3- or 4-membered alkyl or alkenyl chain.

39. A method as defined in claim 32, wherein said cGMP PDE inhibitor has the structure 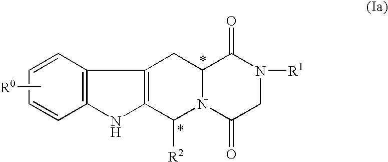
and salts and solvates thereof, in which:
R⁰ represents hydrogen, halogen or C₁₋₆alkyl;
R¹ represents hydrogen, C₁₋₆alkyl, haloC₁₋₆alkyl, C₃₋₈cycloalkyl, C₃₋₈cycloalkyl-C₁₋₃alkyl, arylC₁₋₃alkyl or heteroarylC₁₋₃alkyl; and
R² represents an optionally substituted monocyclic aromatic ring selected from benzene thiophene, furan and pyridine or an optionally substituted bicyclic ring 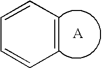
attached to the rest of the molecule via one of the benene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen.

40. A composition as defined in claim 31, wherein said first compound is an α-adrenergic antagonist that is non-selective.

41. A composition as defined in claim 31, wherein said first compound is an α-adrenergic antagonist that is a selective α₁-adrenergic antagonist.

42. A composition as defined in claim 31, wherein said α-adrenergic antagonist is selected from doxazosin, terazosin, abanoquil, prazosin, alfuzosin, indoramin, naftopidil, phentolamine, tamsulosin, trazodone, dapiprazole, phenoxybenzamine, idazoxan, efaroxan, yohimbine, and pharmaceutically acceptable salts thereof.

43. A composition as defined in claim 42, wherein said α-adrenergic antagonist is selected from doxazosin, terazosin, abanoquil, prazosin, and pharmaceutically acceptable salts thereof.

44. A composition as defined in claim 43, wherein said α-adrenergic antagonist is doxazosin, abanoquil, or a pharmaceutically acceptable salt of either.

45. A composition as defined in claim 44, wherein said α-adrenergic antagonist is doxazosin mesylate or abanoquil mesylate.

46. A composition as defined in claim 31, wherein said first compound is doxazosin, abanoquil, or a pharmaceutically acceptable salt of either, and said second compound is sildenafil or a pharmaceutically acceptable salt thereof.

47. A composition as defined in claim 46, wherein said first compound is doxazosin mesylate and said second compound is sidenafil citrate.

48. A composition as defined in claim 46, wherein said first compound is abanoquil mesylate and said second compound is sildenafil citrate.

49. A composition as defined in claim 31, wherein (1) and (2) are selected from the following:
(a) (1) is a selective α₂-adrenergic antagonist and (2) is a cGMP PDE inhibitor;
(b) (1) is a non-selective α-adrenergic antagonist and (2) is a cGMP PDE inhibitor; and
(c) (1) is a selective α₁-adrenergic antagonist (2) is a cGMP PDE inhibitor.

50. A composition as defined in claim 49, wherein said cGMP PDE inhibitor is selective for the PDEᵥ isoenzyme.

51. A composition as defined in claim 49, wherein said α-adrenergic antagonist is selected from doxazosin, terazosin, abanoquil, prazosin, alfuzosin, indoramin, naftopidil, phentolamine, tamsulosin, trazodone, dapiprazole, phenoxybenzamine, idazoxan, efaroxan, yohimbine, and pharmaceutically acceptable salts thereof.

52. A composition as defined in claim 49, which comprises (1) an α-adrenergic antagonist selected from doxazosin, terazosin, abanoquil, prazosin, and pharmaceutically acceptable salts thereof; and (2) sildenafil or a pharmaceutically acceptable salt thereof.

53. A composition as defined in claim 52, wherein said α-adrenergic antagonist (1) is abanoquil, doxazosin or a pharmaceutically acceptable salt of either and (2) is sidenafil citrate.

54. A composition as defined in claim 53, wherein said α-adrenergic antagonist (1) is doxazosin mesylate.

55. A composition as defined in claim 53, wherein said α-adrenergic antagonist (1) is abanoquil mesylate.

56. A composition as defined in claim 31, which is administered orally.

57.-106. (canceled)

107. A kit comprising
(1) a therapeutically effective amount of a first composition comprising a compound selected from α-adrenergic antagonists, plus a pharmaceutically acceptable carrier or diluent, in a first dosage form;
(2) a therapeutically effective amount of a second composition comprising a compound selected from agents which elevate cGMP levels, plus a pharmaceutically acceptable carrier or diluent, in a second dosage form; and
(3) container means for containing said first and second dosage forms.

108.-114. (canceled)

115. A kit as defined in claim 107, wherein said α-adrenergic antagonist is non-selective.

116. A kit as defined in claim 107, wherein said α-adrenergic antagonist is a selective α₁-adrenergic antagonist.

117. A kit as defined in claim 107, wherein said α-adrenergic antagonist is selected from doxazosin, terazosin, abanoquil, prazosin, alfuzosin, indoramin, naftopidil, phentolamine, tamsulosin, trazodone, dapiprazole, phenoxybenzamine, idazoxan, efaroxan, yohimbine, and pharmaceutically acceptable salts thereof salts.

118. A kit as defined in claim 117, wherein said α-adrenergic antagonist is selected from doxazosin, terazosin, abanoquil, prazosin and pharmaceutically acceptable salts thereof.

119. A kit as defined in claim 107, wherein (1) is an β-adrenergic antagonist selected from doxazosin, terazosin, abanoquil, prazosin, and pharmaceutically acceptable salts thereof; and (2) is sildenafil or a pharmaceutically acceptable salt thereof.

120. A kit as defined in claim 119, wherein said α₁-adrenergic antagonist is doxazosin or a pharmaceutically acceptable salt thereof.

121. A kit as defined in claim 119, wherein said sildenafil salt is the citrate.

122. A kit as defined in claim 107, wherein (1) and (2) are each administered orally.

123. A kit as defined in claim 107, adapted for the treatment of male erectile dysfunction or of female sexual dysfunction.

124.-126. (canceled)
